(19) United States
(12) Patent Application Publication
(10) Pub. No.: US 2010/0285085 A1
(43) Pub. Date: Nov. 11, 2010

(54) BALLOON COATING WITH DRUG TRANSFER CONTROL VIA COATING THICKNESS

(75) Inventors: John J. Stankus, Campbell, CA (US); Stephen D. Pacetti, San Jose, CA (US)

Correspondence Address: BAKER BOTTS L.L.P., 30 ROCKEFELLER PLAZA, 44th Floor, NEW YORK, NY, 10112-4498, US

(73) Assignee: Abbott Cardiovascular Systems Inc.

(21) Appl. No.: 12/437,401

(22) Filed: May 7, 2009

(51) Int. Cl. A61L 29/16; A61K 31/436; A61K 31/337; B05D 3/00; A61P 9/10

(52) U.S. Cl. 424/423; 514/291; 514/449; 427/2.3

(57) ABSTRACT

A coated medical device, such a balloon or stent. The coating includes a therapeutic agent having a thickness of the coating is between about 1.5 to 10 μm and less than 30% of the coating remains on the balloon post delivery to a vessel.

### FIELD OF THE INVENTION

The present invention is related to the delivery of drugs from an insertable medical device. More particularly, the present invention relates to a coated balloon having a coating thickness exhibiting improved coating transfer efficiency and/or uptake of therapeutic agent to a blood vessel wall.

### BACKGROUND OF THE INVENTION

Atherosclerosis is a syndrome affecting arterial blood vessels. It is a chronic inflammatory response in the walls of arteries, which is in large part due to the accumulation of lipid, macrophages, foam cells and the formation of plaque in the arterial wall. Atherosclerosis is commonly referred to as hardening of the arteries although the pathophysiology of the disease manifests itself with several different types lesions ranging from fibrotic to lipid laden to calcific. Angioplasty is a vascular interventional technique involving mechanically widening an obstructed blood vessel, typically caused by atherosclerosis.

During angioplasty, a catheter having a tightly folded balloon is inserted into the vasculature of the patient and is passed to the narrowed location of the blood vessel at which point the balloon is inflated to a fixed size using fluid pressures. Percutaneous coronary intervention (PCI), commonly known as coronary angioplasty, is a therapeutic procedure to treat the stenotic coronary arteries of the heart, often found in coronary heart disease. In contrast, peripheral angioplasty, commonly known as percutaneous transluminal angioplasty (PTA), refers to the use of mechanical widening of blood vessels other than the coronary arteries. PTA is most commonly used to treat narrowing of the leg arteries, especially, the iliac, external iliac, superficial femoral and popliteal arteries. PTA can also treat narrowing of veins, and other blood vessels.

Although the blood vessel is often successfully widened by angioplasty, sometimes the treated wall of the blood vessel undergoes vasospasm, or abrubt closure after balloon inflation or dilatation, causing the blood vessel to collapse after the balloon is deflated or shortly thereafter. One solution to such collapse is stenting the blood vessel to prevent collapse. A stent is a device, typically a metal tube or scaffold, that is inserted into the blood vessel after, or concurrently with angioplasty, to hold the blood vessel open.

While the advent of stents eliminated many of the complications of abrupt vessel closure after angioplasty procedures, within about six months of stenting a re-narrowing of the blood vessel often formed, a condition known as restenosis. Restenosis was discovered to be a response to the injury of the angioplasty procedure and is characterized by a growth of smooth muscle cells—analogous to a scar forming over an injury. It was thought that drug eluting stents were the answer to the reoccurrence of the narrowing of blood vessels after stent implantation. A drug eluting stent is a metal stent that has been coated with a drug that is known to interfere with the process of re-narrowing of the blood vessel (restenosis). It was then discovered that a drawback of drug eluting stents was a condition known as late stent thrombosis, which is an event in which blood clots inside the stent. Stent thrombosis, whether acute or late, can be fatal in over one-third of cases.

Drug eluting balloons are believed to be a viable alternative to drug eluting stents in the treatment of atherosclerosis. In a study which evaluated restenosis, and the rate of major adverse cardiac events such as heart attack, bypass, repeat stenosis, or death in patients treated with drug eluting balloons and drug eluting stents, the patients treated with drug eluting balloons experienced only 3.7 percent restenosis and 4.8% MACE (material adverse coronary events) as compared to patients treated with drug eluting stents, in which restenosis was 20.8 percent and 22.0 percent MACE rate. (See, PEPCAD II study, Rotenburg, Germany).

Although drug eluting balloons are a viable alternative, and in some cases appear to have greater efficacy than drug eluting stents as suggested by the PEPCAD II study, drug eluting balloons present unique challenges. In particular, the drug needs to be released from the balloon surface or the coating needs to be transferred to the blood vessel wall when the balloon is expanded inside the blood vessel. For coronary procedures, the balloon is typically inflated for less than one minute, typically about thirty seconds. The balloon may be able to be expanded for a longer period of time for peripheral procedure, however typically even for peripheral procedures the balloon is expanded for less than 5 minutes. Due to the very short duration of contact of the drug coated balloon surface with the blood vessel wall, the balloon coating must exhibit optimal therapeutic agent transfer efficiency and/or efficient drug release during inflation which is within minutes. Thus, there are challenges specific to drug delivery via a drug coated (or drug eluting) balloon because of the necessity of a short inflation time, and therefore time for drug or coating transfer—a challenge not presented by a drug eluting stent, which remains in the patient's vasculature once implanted.

### SUMMARY OF INVENTION

The present invention includes a drug delivery balloon which exhibits improved coating transfer efficiency to the wall of a blood vessel and/or increased uptake of therapeutic agent into a blood vessel wall. Generally, the balloon of the invention has a coating applied to at least a portion of the balloon surface. The coating has a thickness of about 1.5 to about 10 μm. Preferably, the coating has a thickness is of about 2 to about 6 μm. It has surprisingly been found that a drug delivery balloon having such a coating thicknesses exhibits greater coating transfer efficiency and therapeutic uptake.

The coating includes a therapeutic agent and has a thickness between about 1.5 and 10 μm, preferably between about 2 and 6 μm. Surprisingly, less than 30% of the coating remains on the balloon post delivery, inflation and deflation, or post removal from a lumen of a subject. Preferably, less than 20% and more preferably less than 10% of the coating remains on the balloon post delivery, inflation and deflation, or post removal from a lumen of a subject.

In accordance with the invention, various therapeutic agents can be employed. The therapeutic agent can be hydrophobic or hydrophilic. Some non-limiting examples of hydrophobic therapeutic agents include cytostatic drugs, such as zotarolimus everolimus, sirolimus, temsirolimus, biolimus, deforolimus, novolimus, and myolimus. Other antiproliferative drugs such as paclitaxel, protaxel and taxanes may be also be use in addition to other therapeutic agents. In one embodiment, the dosage of therapeutic agent is about 15 ug/cm² to about 600 ug/cm².

The coating can further include an excipient, however an excipient is not required. The excipient is preferably less than 75% or less than 50% by weight of the coating. The excipient can have hydrophilic properties and binder properties. Various excipients can be used, such as polysorbates including Tween 20 and Tween 80. Other examples include polyethylene glycol and polyvinyl pyrrolidone (PVP). Preferably, the PVP is not substantially cross-linked, and is not a hydrogel. In one embodiment, the PVP has a molecular weight of less than 60 kilodaltons. In yet another embodiment, the PVP has a molecular weight of less than 30 kilodaltons. In accordance with one embodiment, polyethylene glycol has a molecular weight less than 1000 daltons.

The coating can further includes a plasticizer, such as but not limited to glycerol, polyethylene glycol, propylene glycol, tween20, dimethylsulfoxide, N-methylpyrrolidone, benzyl alcohol, or benzyl benzoate. For example, the coating can include zotarolimus, PVP, and glycerol. In one embodiment, the weight ratio of the zotarolimus:PVP:glycerol is about 20:1 to 1:2 for zotarolimus:PVP, preferably, is about 1:1 to 1:0.1 for PVP:glycerol and more preferably is about 2:1:0.4 for zotarolimus:PVP:glycerol. In another embodiment, the coating includes zotarolimus and a non-ionic contrast agent, such as but not limited to an iopromide. In one embodiment, the iopromide is Ultravist. The weight ratio of the zotarolimus:non-ionic contrast agent is about 10:1 to 1:10 and more preferably about 2:1, such as 1.95:1.

Preferably, the coated balloon is disposed on a catheter body for insertion of the drug delivery balloon to the vasculature of a patient. The catheter can include an elongate tubular member having a proximal end, a distal end and a lumen there between. In one embodiment, the catheter has an over-the-wire configuration. In another embodiment, catheter has a rapid exchange configuration.

In accordance with another aspect of the invention, a coated medical device is provided, such as a balloon including a stent. The medical device includes an expandable member having a surface and a coating applied to at least a portion of the surface of the expandable member. The coating comprises a therapeutic agent and an excipient and has a thickness of about 1.5 to 10 μm.

In yet another aspect of the invention, a method of manufacturing a drug delivery device is provided. The drug delivery device for example is a balloon. In this regard, the method includes applying a coating to at least a portion of an expandable member to define a thickness of about 1.5 to 10 um, and preferably from 2 to 6 um, and disposing the expandable member on a catheter. The method can further include the step of preparing a pre-coating mixture for example by mixing a therapeutic agent and an excipient, and conditioning the pre-coating to form a porous coating by a phase inversion technique. Additionally, or alternatively, the method can include the step of creating a coating to which a porogen is added to define a porous coating for application to the medical device.

In one embodiment, the porous coating is created by phase inversion techniques. In another embodiment, the porous coating is created by introduction of a porogen to a mixture including a therapeutic agent to be applied to the delivery device. In one embodiment, the porogen is removed from the coating prior to application of the coating to the delivery device.

It is to be understood that both the foregoing description is exemplary and is intended to provide further explanation of the invention claimed to a person of ordinary skill in the art. The accompanying drawings are included to illustrate various embodiments of the invention to provide a further understanding of the invention. The exemplified embodiments of the invention are not intended to limit the scope of the claims.

### BRIEF DESCRIPTION OF DRAWINGS

FIG. 1 depicts one embodiment of a medical device of the invention;

FIG. 2 is a graph illustrating the results from a comparative study of drug delivery balloons and coating transfer efficiency in a porcine coronary and mammary pharmacokinetic model;

FIG. 3 is a graph illustrating percent drug remaining on post delivery balloons as a function of theoretical coating thicknesses of drug delivery balloons having varied formulations;

FIG. 4 is a graph illustrating therapeutic agent and percent initial balloon dose remaining in tissue after delivery in a porcine coronary and mammary pharmacokinetic model using an embodiment of the present invention;

FIG. 5 depicts one embodiment of a medical device of the invention.

### DETAILED DESCRIPTION

Reference will now be made in detail to the present embodiments of the invention, an example of which is illustrated in the accompanying figures. The invention will be described in conjunction with the detailed description of the device. However, no intent to limit the scope of the invention to the specific embodiments described exists.

The device and method of the invention may be used for treating the lumen of a patient. In particular, the invention is particularly suited for treatment of the cardiovascular system of a patient, such as performance of angioplasty and/or delivery of a coated expandable medical device, such as a stent, filter, or coil in the coronary or peripheral blood vessels.

In accordance with one aspect of the invention, a balloon for delivering a therapeutic agent is provided. The balloon includes a body having a working portion disposed between distal and proximal ends of the balloon, such as between first and second cone portions, and a coating applied to at least a portion of the balloon. The coating includes a therapeutic agent and has a thickness between about 1.5 to 10 μm, and more preferably, a thickness of about 2 to about 6 μm.

In one embodiment, less than 10% of the coating remains on the balloon or medical device post delivery into a lumen of a subject. That is, at least 90% of the coating is delivered from the balloon or medical device. In another embodiment, less than 30% of the coating remains on the balloon after inflation and deflation in the lumen of a subject. In yet another embodiment, less than 30% of the coating remains on the balloon or expandable medical device post removal of the balloon or medical device from the lumen of the subject. Preferably, less than 20% of the coating remains on the balloon or medical device post delivery, inflation and deflation, and/or removal from a lumen of a subject. More preferably, less than 10% of the coating remains on the balloon or medical device post delivery, inflation and deflation, and/or removal from a lumen of a subject.

The therapeutic agent can be any therapeutic agent. However, preferably, the therapeutic agent is an antiproliferative or a cytostatic drug. The term “cytostatic” as used herein means a drug that mitigates cell proliferation but allows cell migration. For the purpose of illustration without limitation, the cytostatic drug includes zotarolimus, everolimus, sirolimus, deforolimus, biolimus, myolimus, novolimus, and temsirolimus. The term “antiproliferative” as used herein means a drug used to inhibit cell growth, such as chemotherapeutic drugs. Some non-limiting examples of antiproliferative drugs include taxanes, paclitaxel, and protaxel.

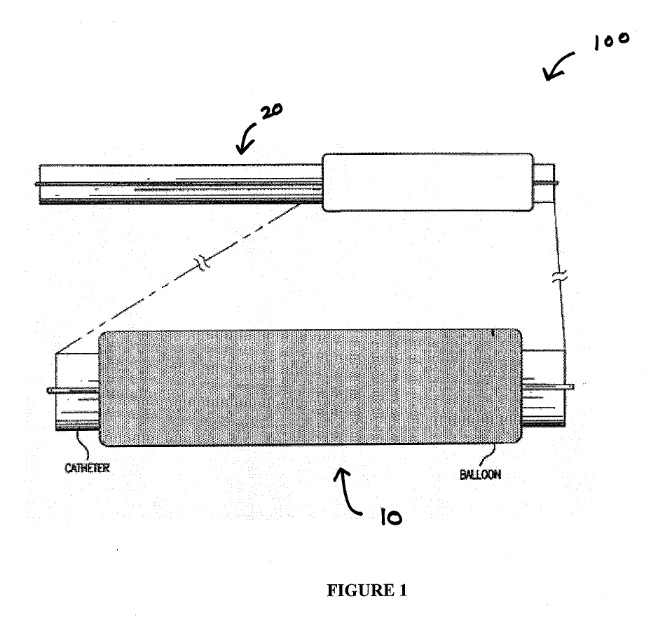

Referring to FIG. 1, a device 100 is provided drug delivery balloon 10 that exhibits improved coating transfer from the balloon and/or therapeutic agent uptake to a blood vessel wall is provided. In one embodiment, the balloon 10 is disposed on a catheter 10, as shown in FIG. 1. In this regard, it has been surprisingly discovered that a balloon having a coating thickness of about 1.5 to 10 μm and preferably 2 to 6 μm exhibits improved coating transfer efficiency. In one embodiment, less than 30% of the initial coating remains on the balloon post delivery to a lumen in a subject. In another embodiment, less than 30% of the coating remains on the balloon or at least a portion of the balloon post inflation and deflation in a lumen of a subject. In yet another embodiment, less than 30% of the coating remains on the balloon post removal from a subject. Accordingly, more than 70% of the coating transfers from the balloon to the subject. Preferably, less than 20% of the coating remains on the balloon, and more preferably less than 10% of the coating remains on the balloon.

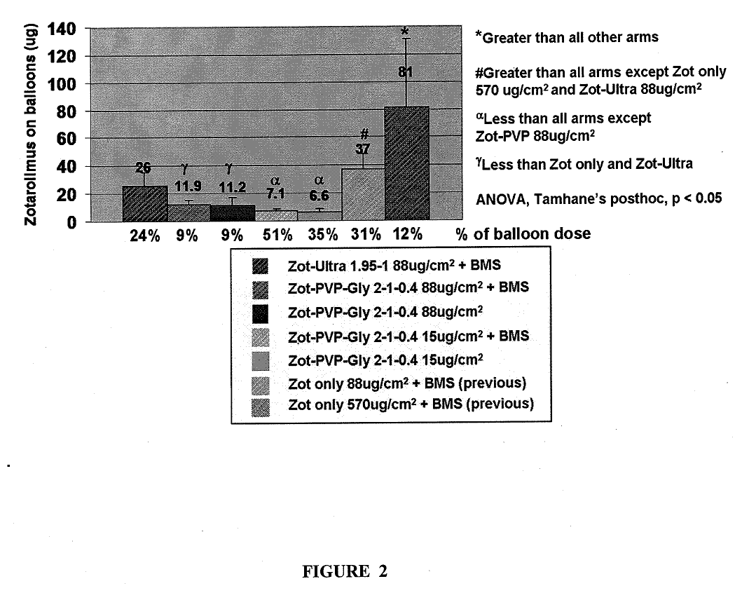

FIG. 2 shows the results from a comparative study in which seven different coated balloons were delivered to healthy porcine coronary or mammary arteries in pharmacokinetic models. The coating formulations are tabulated in Table 1.

TABLE 1

| | | Dosage of |
|---|---|---|
| | | Therapeutic |
| Balloon | Formulation | Agent |
| 1 | Zotarolimus:Ultravist (1.95:1 weight ratio) | 88 μg/cm² |
| 2 | Zotarolimus:PVP:Glycerol (2:1:0.4 weight ratio) | 88 μg/cm² |
| 3 | Zotarolimus:PVP:Glycerol (2:1:0.4 weight ratio) | 88 μg/cm² |
| | | (no stent) |
| 4 | Zotarolimus:PVP:Glycerol (2:1:0.4 weight ratio) | 15 μg/cm² |
| 5 | Zotarolimus:PVP:Glycerol (2:1:0.4 weight ratio) | 15 μg/cm² |
| | | (no stent) |
| 6 | Zotarolimus | 88 μg/cm² |
| 7 | Zotarolimus | 570 μg/cm² |

All of the coatings include a therapeutic agent. Most of the coating formulations include excipients or different doses of therapeutic agent to achieve varied coating thickness on the balloons. The drug delivery balloons were inserted and inflated for 30 seconds in the animal model. Thereafter, the drug delivery balloons were withdrawn and then the percentage of the initial drug dosage remaining on the balloon surface was calculated. The remaining drug on each of the balloons was assayed by extraction of the balloons in an organic solvent mixture followed by analysis using high pressure liquid chromatography (HPLC).

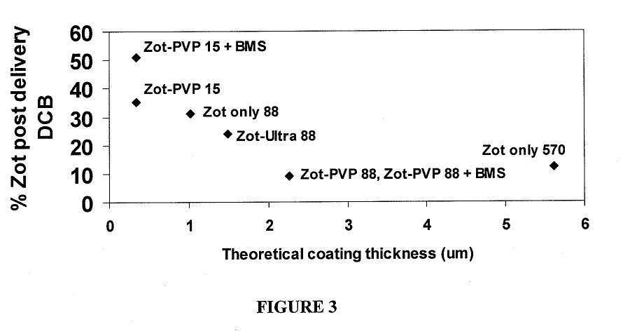

It was surprisingly found that the balloons having thicker coatings exhibited greater coating transfer efficiency from the balloon to the blood vessel wall. In particular, as depicted in FIG. 2, Balloons 6 and 7 (counting from left to right) are each coated with pure zotarolimus. The zotarolimus coating applied to Balloon 7 has a dose density of 570 μg/cm² of zotarolimus, and the coating applied to Balloon 6 has 88 μg/cm² of zotarolimus. As shown in FIG. 3, the theoretical thicknesses for the coatings was calculated to be about 1 μm for Balloon 6 and about 6 μm for Balloon 7. This theoretical thickness were calculated based on the mass and density of the coating and balloon surface area via the formula:

$$T = \frac{W}{A ρ} = \frac{{V}_{coating}}{A}$$

where T=average coating thickness
   W=coating mass
   A=coated balloon area
   ρ=coating density (assumed 1.1 gm/cm³)
   V_{coating}=coating volume
      As shown, Balloon 7 has a greater coating transfer efficiency than does Balloon 6. In particular, the percentage of coating transfer for Balloon 6 is 69%, whereas the balloon coating transfer for Balloon 7 is 88%.

Likewise, Balloons 2 and 3 each have coating formulations comprising zotarolimus, PVP, and glycerol. The dosage of zotarolimus is 88 μg/cm² and the drug:PVP:Glycerol is in a ratio of 2:1:0.4. In contrast, Balloons 4 and 5 also have a coating of zotarolimus, PVP, and glycerol in a 2:1:0.4 ratio. However, the dosage of zotarolimus in Balloons 4 and 5 is 15 μg/cm². As shown in FIG. 3, the theoretical coating thicknesses of Balloons 2 and 3 are 2.25 μm, whereas the theoretical coating thicknesses for Balloons 4 and 5 are 0.5 μm. Balloons 2 and 3 both exhibit over 90% coating transfer efficiency, while Balloons 4 and 5 exhibit less than 65% coating transfer, as shown in FIG. 2. Thus, the balloons having thicker coatings resulted in improved coating transfer efficiency.

Referring to FIG. 2, Balloon 1 has a coating formulation of zotarolimus and Ultravist in a ratio of 1.95:1 (w/w). The theoretical coating thickness of Balloon 1 is about 1.5 μm, as shown in FIG. 3. Balloon 1 exhibited a coating transfer of about 76%, which is a greater coating transfer efficiency than the Balloons 4 and 5 having a coating thickness of about 0.5 μm, but a lesser coating transfer efficiency than Balloons 2 and 3 which exhibited 90% coating transfer efficiency.

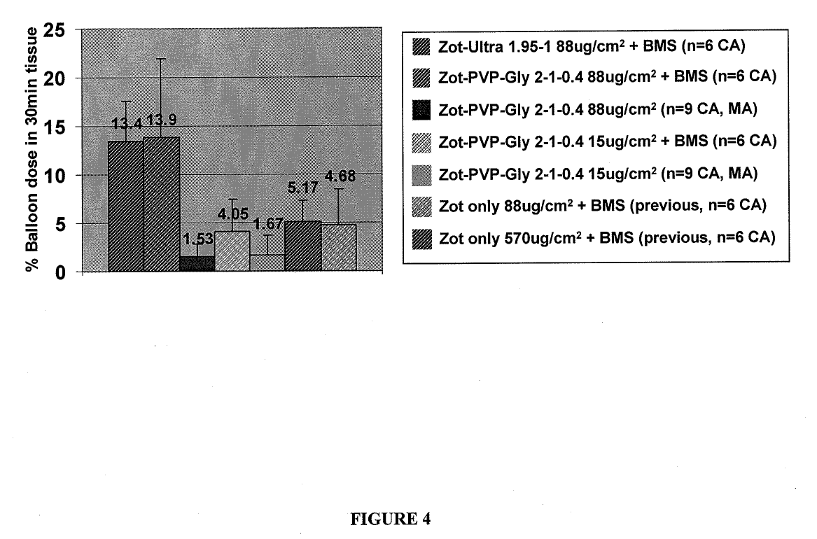

In another aspect of the invention, a drug delivery balloon is provided which exhibits improved tissue uptake of therapeutic agent. FIG. 4 shows the results from a comparative study in which various drug delivery balloons having the formulations of Table 1 were inserted and inflated in porcine coronary and mammary artery pharmacokinetic models. The drug delivery balloons were inserted via femoral access and delivered to either the LCX, LAD, RCA, LIMA or RIMA arteries for a thirty second inflation. After deflation of the balloon and removal, the balloons were clipped and frozen until HPLC analysis. The percent of zotarolimus dose per the original balloon dose transferred to the tissue 30 minutes after balloon inflation is depicted in the graph of FIG. 4.

As shown in FIG. 4, Balloon 2 and Balloon 4 both have formulations of zotarolimus:PVP:glycerol. The coatings differ in that Balloon 4 has zotarolimus in an amount of 15 μg/cm² and Balloon 2 has zotarolimus in an amount of 88 μg/cm². Consequently, the coating of Balloon 2 is thicker than the coating of Balloon 4. As shown in FIG. 4, Balloon 2 exhibits greater tissue uptake of zotarolimus than does Balloon 4. Thus, it appears drug delivery balloons having a thicker coating improves drug uptake into the tissue of the vessel wall.

Further, it was surprisingly found that the tissue uptake has greater improvements when the drug delivery balloon includes a stent crimped on the balloon. In this regard, comparison of Balloon 2 and Balloon 3, each of which have identical coating formulations, exhibited different drug uptake into the tissues of the vessel walls. In particular, Balloon 2 which includes a bare metal stent crimped on the balloon during delivery exhibited greater than six-fold increase in zotarolimus tissue uptake than did Balloon 3, which has no stent disposed on the drug delivery balloon.

Likewise, Balloon 4 and Balloon 5 each include identical coating formulations, except that Balloon 4 further includes a bare metal stent disposed on the balloon and balloon 5 has no stent. As shown in FIG. 4, the inclusion of a stent crimped on the Balloon 4 resulted in a greater than two-fold increase in zotarolimus uptake by the tissue as compared to Balloon 5. Thus, in addition to coating thicknesses, the inclusion of a bare metal stent disposed on the drug delivery balloon improves tissue uptake of therapeutic agent. Thus, in another aspect of the invention, a drug delivery balloon is provided which exhibits improved tissue uptake of therapeutic agent in one aspect of the invention. The drug delivery balloon comprises a coating applied to at least a portion of the balloon surface and a stent disposed on balloon. In this regard, the stent can be a bare metal stent, a coated stent or a drug eluting stent.

In accordance with the invention, the coating can be applied to a medical device by processes such as dip-coating, pipette coating, syringe coating, air assisted spraying, electrostatic spraying, piezoelectric spraying, electrospinning, direct fluid application, or other means as known to those skilled in the art. The coating may contain the drug homogeneously dissolved or encapsulated in particles. The coating can be applied over at least a portion or the entirety of the balloon or medical device. By way of example, and not limitation, certain coating processes that may be used with the instant invention are described in U.S. Pat. No. 6,669,980 to Hansen; U.S. Pat. No. 7,241,344 to Worsham; and U.S. Publication No. 2004/0234748 to Stenzel, the entire disclosures of which are hereby incorporated by reference. In accordance with one embodiment of the invention, the medical device is a balloon and the coating can be applied to either a folded or inflated balloon. Coating characteristics are affected by process variables. For example, for a dip-coating process, coating quality and thickness can vary as an effect of variables such as number of dips, rate of withdrawal, and depth of dips along with drying time and temperature.

In accordance with another aspect of the invention, a method of manufacturing a drug delivery device is provided. The drug delivery device can be for example a balloon or a stent. The method includes applying a coating including an effective amount of a therapeutic agent to an expandable member to define a coating thickness of about 1.5 to about 10 μm, and disposing the expandable member on a catheter. In an alternative embodiment, providing a catheter including an expandable member; and applying a coating including an effective amount of a therapeutic agent to the expandable member to define a coating thickness of about 1.5 to about 10 μm. The catheter includes an elongate shaft having a proximal end, a distal end and at least one lumen therebetween. Preferably, the catheter includes a multilumen shaft such as an inflation lumen and a guidewire lumen. In this regard, multilumen can be arranged in a coaxial or side-by-side configuration. Further, the catheter can be configured as a rapid exchange catheter or an over-the-wire catheter.

The method can further include the step of preparing the coating, during which the preparation step includes mixing a therapeutic agent, such as an effective amount of a therapeutic agent, and an excipient to form a precoating, and conditioning the precoating by a phase inversion technique to define a porous coating for application to the expandable member. Alternatively, or additionally, the method can include defining a porous coating by adding a porogen to the coating or preparing the coating by inclusion of a porogen, as described below.

In accordance with the invention, the coating thickness applied to a medical device or a balloon is controlled. Various techniques are available to control the coating thickness for a drug delivery balloon. For the purpose of illustration but not limitation, the coating thickness can be controlled by changing: (1) drug dose per unit of balloon surface area, (2) percent solids of drugs and excipients in the coating solution, (3) ratio of therapeutic agent to excipients in the drug formulation, (4) changing the surface area of coating per a certain dose and formulation, (5) adding porosity or void volume of the coating, or (6) particulars of the coating process such as coating method, drying rate and solvent used.

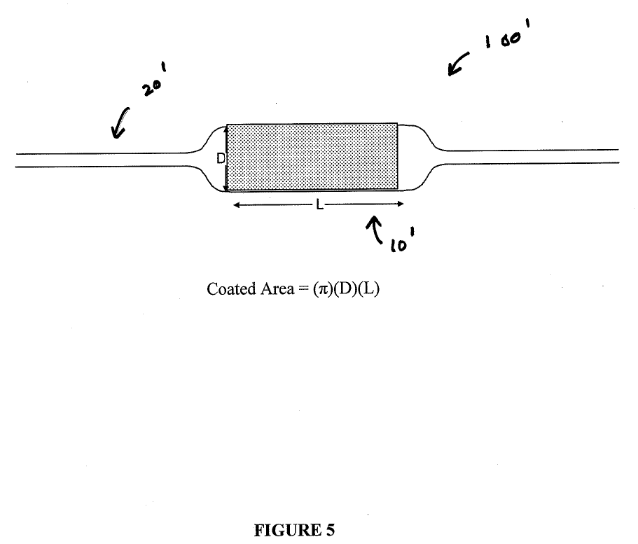

In one embodiment, the coating thickness is controlled for a given therapeutic agent dose and formulation. For example and for the purpose of illustration but not limitation, FIG. 5 shows that the coated area for drug delivery balloon 10 can be calculated by the following equation: Coated Area=(π)(D)(L); where D is the diameter of the balloon and L is the working length or coated length of the balloon.

For example, the surface area may be reduced by decreasing length (L) of the balloon for a particular therapeutic agent dose and formulation. Rather than decreasing the working area of the balloon that is coated, the balloon may be coated by a series of bands wrapping around the balloon, or stripes running along the length of the balloon. Many other patterns are possible such as checkerboard or a plurality of dots. In all of these cases, the amount of drug dissolution from the balloon, rate of drug dissolution, or coating transfer to the vessel wall will be increased via an increase in coating thickness.

Other means to increase the coating thickness include: (1) increasing the therapeutic agent dose, and (2) increasing the amount of excipient for a given drug dose. While increasing the dose will render the coating more prone to fracture, during inflation there is an upper limit on the amount of therapeutic agent that can be used so as not to exceed the no observable adverse effect level (“NOAEL”), which is based on systemic drug exposure and available toxicological data for the drug.

In addition, a porous coating of the same dose would have a larger coating thickness. There are many methods to create a porous, open celled coating such as (1) incorporation of a porogen into the coating, which is subsequently leached out after the coating process (e.g., salt leaching) and (2) use of a coating which undergoes phase inversion (e.g., thermal induced phase separation). Phase inversion is a process that creates porous structures. Phase inversion either starts with a homogenous single phase solution (Sol 1) which at some point before gelation undergoes a transition into a heterogeneous solution of molecular aggregates consisting of two interdispersed liquid phases (Sol 2), or it starts with a heterogeneous solution of molecular aggregates consisting of two interdispersed liquid phases (Sol 2).

Phase inversion can be accomplished by use of a solvent and excipient blends in a drying process, a thermal process where the polymer is only soluble at an elevated temperature in the solvent, or a wet process where a dense coating is subsequently exposed to additional solvent processing. The drying process is most applicable to coatings containing a drug. A simple concept is to dissolve the drug and excipients in a solvent blend where the faster evaporating solvent is compatible solvents for the polymer/drug. Other examples of phase inversion techniques to produce porous surfaces include lyophilization, high pressure gas foaming, solid freeform fabrication, fiber bonding of extruded microfibers and fiber based electrospinning of micro- or nanofibers.

In accordance with the invention, the balloon is a polymeric expandable balloon. Various polymers may be selected for the formation of the balloon, as would be known in the art. For example, the polymeric material may be may be a compliant, non-compliant or semi-compliant polymeric material or polymeric blend.

In one embodiment, the polymeric material is compliant such as but not limited to a polyamide/polyether block copolymer (commonly referred to as PEBA or polyether-block-amide). Preferably, the polyamide and polyether segments of the block copolymers may be linked through amide or ester linkages. The polyamide block may be selected from various aliphatic or aromatic polyamides known in the art. Preferably, the polyamide is aliphatic. Some non-limiting examples include nylon 12, nylon 11, nylon 9, nylon 6, nylon 6/12, nylon 6/11, nylon 6/9, and nylon 6/6. Preferably, the polyamide is nylon 12. The polyether block may be selected from various polyethers known in the art. Some non-limiting examples of polyether segments include poly(tetramethylene glycol), tetramethylene ether, polyethylene glycol, polypropylene glycol, poly(pentamethylene ether) and poly(hexamethylene ether). Commercially available PEBA material may also be utilized such as for example, PEBAX® materials supplied by Arkema (France). Various techniques for forming a balloon from polyamide/polyether block copolymer are known in the art. One such example is disclosed in U.S. Pat. No. 6,406,457 to Wang, the disclosure of which is incorporated by reference.

In another embodiment, the balloon material is formed from polyamides. Preferably, the polyamide has substantial tensile strength, be resistant to pin-holing even after folding and unfolding, and be generally scratch resistant, such as those disclosed in U.S. Pat. No. 6,500,148 to Pinchuk, the disclosure of which is incorporated herein by reference. Some non-limiting examples of polyamide materials suitable for the balloon include nylon 12, nylon 11, nylon 9, nylon 69 and nylon 66. Preferably, the polyamide is nylon 12. In yet another embodiment, the balloon is composed of several different layers, each a one a different polyamide or polyamide/polyether block copolymer.

In another embodiment, the balloon may be formed a polyurethane material, such as TECOTHANE® (Thermedics). TECOTHANE® is a thermoplastic, aromatic, polyether polyurethane synthesized from methylene disocyanate (MDI), polytetramethylene ether glycol (PTMEG) and 1,4 butanediol chain extender. TECOTHANE® grade 1065D is presently preferred, and has a Shore durometer of 65D, an elongation at break of about 300%, and a high tensile strength at yield of about 10,000 psi. However, other suitable grades may be used, including TECOTHANE® 1075D, having a Shore D of 75. Other suitable compliant polymeric materials include ENGAGE® (DuPont Dow Elastomers (an ethylene alpha-olefin polymer) and EXACT® (Exxon Chemical), both of which are thermoplastic polymers. Other suitable compliant materials include, but are not limited to, elastomeric silicones, latexes, and urethanes. The compliant material may be cross linked or uncrosslinked, depending upon the balloon material and characteristics required for a particular application. The presently preferred polyurethane balloon materials are not crosslinked. However, other suitable materials, such as the polyolefinic polymers ENGAGE® and EXACT®, are preferably crosslinked. By crosslinking the balloon compliant material, the final inflated balloon size can be controlled. Conventional crosslinking techniques can be used including thermal treatment and E-beam exposure. After crosslinking, initial pressurization, expansion, and preshrinking, the balloon will thereafter expand in a controlled manner to a reproducible diameter in response to a given inflation pressure, and thereby avoid overexpanding the stent (when used in a stent delivery system) to an undesirably large diameter. In one embodiment, the balloon is formed from a low tensile set polymer such as a silicone-polyurethane copolymer. Preferably, the silicone-polyurethane is an ether urethane and more specifically an aliphatic ether urethane such as PURSIL AL 575A and PURSIL AL10, (Polymer Technology Group), and ELAST-EON 3-70A, (Elastomedics), which are silicone polyether urethane copolymers, and more specifically, aliphatic ether urethane cosiloxanes. In an alternative embodiment, the low tensile set polymer is a diene polymer. A variety of suitable diene polymers can be used such as but not limited to an isoprene such as an AB and ABA poly(styrene-block-isoprene), a neoprene, an AB and ABA poly(styrene-block-butadiene) such as styrene butadiene styrene (SBS) and styrene butadiene rubber (SBR), and 1,4-polybutadiene. Preferably, the diene polymer is an isoprene including isoprene copolymers and isoprene block copolymers such as poly(styrene-block-isoprene). A presently preferred isoprene is a styrene-isoprene-styrene block copolymer, such as Kraton 1161K available from Kraton, Inc. However, a variety of suitable isoprenes can be used including HT 200 available from Apex Medical, Kraton R 310 available from Kraton, and isoprene (i.e., 2-methyl-1,3-butadiene) available from Dupont Elastomers. Neoprene grades useful in the invention include HT 501 available from Apex Medical, and neoprene (i.e., polychloroprene) available from Dupont Elastomers, including Neoprene G, W, T and A types available from Dupont Elastomers.

In accordance with the invention, the balloon can be composed of a single polymeric layer, or alternatively, can be a multilayered balloon, such as those described in U.S. Pat. No. 5,478,320 to Ishida, U.S. Pat. No. 5,879,369 to Trotta, or U.S. Pat. No. 6,620,127 to Lee, the disclosures of which are incorporated herein by reference.

In one embodiment, the outer surface of the balloon is textured. In this regard, the balloon surface may include a roughened surface, voids, spines, or microcapsules or a combination thereof, as will be described below.

In one embodiment of the invention, the balloon is formed of a porous elastomeric material having at least one void formed in the wall of the balloon surface. The entire cross section of the balloon may contain a plurality of voids. Alternatively, the plurality of void may be distributed along select portions of the balloon outer surface. For example and not limitation, the plurality of voids can be distributed only along only the working section of the balloon. The voids define an open space within the outer surface of the balloon. Preferably, the therapeutic agent is dispersed within the space defined by the plurality of voids across the cross section of the balloon outer surface.

In operation, the therapeutic agent is released or is expelled from the pores upon inflation of the balloon. In this regard, the durometer of the polymeric material of the balloon surface and in particular the depression of the void is sufficiently flexible to allow for expulsion of the therapeutic agent and/or coating contained within the plurality of voids upon inflation of the balloon. The expelled coating with therapeutic agent is released into the vessel lumen or into the tissue surrounding and contacting the inflated balloon.

In another embodiment, as embodied herein, the balloon includes protrusions configured to contact or penetrate the arterial wall of a vessel upon inflation of the balloon. A coating containing therapeutic agent is disposed on the protrusions and when inflated the coating and/or therapeutic agent coats the tissue of the arterial wall. Alternatively, the balloon may include two concentric balloons in a nesting configuration. The coating with therapeutic agent is disposed between the two concentric balloons. Thus, the space between the two concentric balloons; one being an interior balloon and the other being an exterior balloon, acts as a reservoir. In this regard, the protrusions may include apertures for expulsion of the coating and/or therapeutic agent upon inflation of the interior and exterior concentric balloons. For example, as described in U.S. Pat. No. 6,991,617 to Hektner, the disclosure of which is incorporated herein by reference thereto. In another embodiment, the balloon may include longitudinal protrusions configured to form ridges on the balloon surface. As described in U.S. Pat. No. 7,273,417 to Wang, the disclosure of which is incorporated herein by reference, the ridges can be formed of filaments spaced equidistantly apart around the circumference of the balloon. However, a larger or smaller number of ridges can alternatively be used. The longitudinal ridges can be fully or partially enveloped by the polymeric material of the balloon.

In yet another embodiment of the invention, the balloon may include microcapsules on its outer surface. In this regard, the microcapsules are configured to encompass the coating and/or therapeutic agent. Upon inflation of the balloon the microcapsules located on the surface of the balloon contact the tissue of the arterial wall. Alternatively, the microcapsules may be formed in the wall of the balloon surface. The coating and/or therapeutic agent may be released from the microcapsules by fracturing of the microcapsules and/or diffusion from the microcapsule into the arterial wall. The microcapsules may be fabricated in accordance with the methods disclosed in U.S. Pat. No. 5,1023,402 to Dror or U.S. Pat. No. 6,129,705 to Grantz and the patents referenced therein, each of which is incorporated herein by reference.

In accordance with another aspect of the invention, if desired, a protective sheath may be utilized to protect the coating from being rubbed off of the balloon during the movement of the coated balloon through the body lumen. The sheath is preferably made from an elastic and resilient material which conforms to the shape of the balloon and in particular is capable of expanding upon inflation of the balloon. The sheath preferably includes apertures along a portion thereof. In operation, the inflation of the balloon causes the apertures of the sheath to widen for release of the coating and/or therapeutic agent to the tissue of the arterial wall. Preferably, the sheath has a thickness less than 10 mils. However, other thicknesses are possible.

In another embodiment, the sheath has at least one longitudinal line of weakness allowing the sheath to rupture upon inflation of the balloon and the release of the coating and/or therapeutic agent onto the tissue of the arterial wall of the vessel. Preferably, the sheath is formed from polymeric material known to be suitable for use in balloon catheters. Preferably, the sheath material is an elastomeric material which will also spring back when it splits to expose more of the body lumen to the coating. The line of weakness could be provided by various techniques known in the art. However, one non-limiting examples include perforating the sheath material. In operation, the sheath is placed over the coated balloon while in the deflated state. When the coated balloon inflated, the sheath is expanded to the extent that it exceeds its elastic limit at the line of weakness and bursts to expose and therefore release the coating and/or therapeutic agent to the tissue of the arterial wall or vessel lumen. For example, see U.S. Pat. No. 5,370,614 to Amundson, the disclosure of which is incorporated by reference.

In accordance with another aspect of the invention, a coated medical device is provided. The medical device comprises an expandable member having a surface and a coating having a thickness of about 2 to about 6 um is applied to the surface of the expandable member. The. The coating has a thickness of about 2 to about 6 um. For example can be a stent.

What is claimed is:

1. A balloon for delivering a therapeutic agent to a vessel wall, the balloon comprising:
a body having a working portion disposed between distal and proximal ends thereof; and
a coating applied to at least a portion of the balloon, wherein the coating includes a therapeutic agent and has a thickness between about 1.5 to 10 μm.

2. The balloon of claim 1, wherein the thickness of the coating is between 2 to 6 μm.

3. The balloon of claim 1, wherein less than 30% of the coating remains on at least a portion of the balloon post delivery to a lumen of a subject.

4. The balloon of claim 1, wherein less than 30% of the coating remains on at least a portion of the balloon post inflation and deflation in a lumen of a subject.

5. The balloon of claim 1, wherein less than 30% of the coating remains on at least a portion of the balloon post removal of the balloon from a subject.

6. The balloon of claim 1, wherein the coating further includes a porogen.

7. The balloon of claim 1, wherein the therapeutic agent is zotarolimus.

8. The balloon of claim 1, wherein the therapeutic agent is paclitaxel.

9. The balloon of claim 1, wherein the therapeutic agent is selected from the group consisting of everolimus, sirolimus, deforolimus, biolimus, myolimus, novolimus, and temsirolimus.

10. The balloon of claim 1, wherein the coating further includes an excipient.

11. The balloon of claim 10, wherein the excipient is less than 75% by weight of the coating.

12. The balloon of claim 10, wherein the excipient is less than 50% by weight of the coating.

13. The balloon of claim 10, wherein the excipient is polyethylene glycol.

14. The balloon of claim 10, wherein the excipient is a polysorbate.

15. The balloon of claim 10, wherein the excipient is a binder.

16. The balloon of claim 15, wherein the binder is PVP, and further wherein the PVP is not a hydrogel.

17. The balloon of claim 1, wherein the coating further includes a plasticizer.

18. The balloon of claim 17, wherein the plasticizer is glycerol, polyethylene glycol, propylene glycol, tween20, dimethylsulfoxide, N-methylpyrrolidone, benzyl alcohol, or benzyl benzoate.

19. The balloon of claim 18, wherein the polyethylene glycol has a molecular weight less than 1000 daltons.

20. The balloon of claim 1, wherein the coating consists of zotarolimus, PVP, and glycerol.

21. The balloon of claim 20, wherein the ratio of zotarolimus:PVP is from about 20:1 to 1:2.

22. The balloon of claim 20, wherein the ratio of PVP:glycerol is from about 1:1 to 1:0.1.

23. The balloon of claim 20, wherein the ratio of zotarolimus:PVP:glycerol is 2:1:0.4.

24. The balloon catheter of claim 1, wherein the coating consists of zotarolimus and a non-ionic contrast agent.

25. The balloon of claim 24, wherein the weight ratio of zotarolimus: non-ionic contrast agent is about 10:1 to about 1:10.

26. The balloon of claim 25, wherein the non-ionic contrast agent is iopromide and further wherein the weight ratio of zotarolimus:iopromide is about 2:1.

27. The balloon catheter of claim 1, wherein a stent is disposed on the balloon.

28. A coated medical device comprising:
an expandable member having a surface;
a coating applied to at least a portion of the surface of the expandable member, the coating comprising a therapeutic agent and an excipient, wherein the coating has a thickness of about 2 to 6 μm.

29. The coated medical device of claim 28, wherein the medical device is a balloon.

30. The coated medical device of claim 28, wherein the therapeutic agent is selected from the group consisting of: zotarolimus, everolimus, sirolimus, biolimus, deforolimus, novolimus, myolimus, and temsirolimus.

31. The coated medical device of claim 28, wherein the therapeutic agent is paclitaxel, protaxel, or a taxane.

32. The coated medical device of claim 28, wherein the coating comprises zotarolimus, PVP, and glycerol.

33. The coated medical device of claim 32, wherein zotarolimus has a dosage of about 15 μg/cm² to about 600 ug/cm².

34. The coated medical device of claim 32, wherein the ratio of zotarolimus:PVP:glycerol is about 2:1:0.4.

35. A method of manufacturing a drug delivery device comprising:
providing a catheter including an expandable member; and
applying a coating including an effective amount of a therapeutic agent to the expandable member to define a coating thickness of about 1.5 to about 10 μm.

36. The method of claim 35, wherein the coating applied to the expandable member has a thickness of about 2 to 6 μm.

37. The method of claim 35, wherein the coating includes a porogen.

38. The method of claim 35, wherein the catheter includes an elongate shaft having a proximal end, a distal end and at least one lumen therebetween, the expandable member disposed proximate the distal end of the elongate shaft.

39. The method of claim 35, wherein the expandable member is a balloon.

40. The method of claim 35, wherein the expandable member includes a stent.

41. The method of claim 35, further including the step of preparing the coating, the preparing step including mixing a therapeutic agent and an excipient to form a precoating and conditioning the precoating to form a porous coating by a phase inversion technique.
